SEQUENCE LISTING (119 sequences)

SEQ ID NO: 1 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - DNA, 17 bp, Artificial Sequence
  Description of Artificial Sequence: Synthetic primer
gccgaacgcaccggagg

SEQ ID NO: 3 - DNA, 17 bp, Artificial Sequence
  Description of Artificial Sequence: Synthetic primer
gccgaacgcaccggggg

SEQ ID NO: 4 - DNA, 17 bp, Artificial Sequence
  Description of Artificial Sequence: Synthetic primer
gccgaacgcaccgaagg

SEQ ID NO: 5 - DNA, 17 bp, Artificial Sequence
  Description of Artificial Sequence: Synthetic primer
gccgaacgcaccggtgg

SEQ ID NO: 6 - DNA, 17 bp, Artificial Sequence
  Description of Artificial Sequence: Synthetic primer
gccgaacgcaccgcagg

SEQ ID NO: 7 - DNA, 19 bp, Artificial Sequence
  Description of Artificial Sequence: Synthetic primer
  modified_base (17)..(17): t-bb-dA
gtgtcgaacgcaccggagg

SEQ ID NO: 8 - DNA, 23 bp, Artificial Sequence
  Description of Artificial Sequence: Synthetic primer
agcctcttacacccagtggagaa

SEQ ID NO: 9 - DNA, 10 bp, Artificial Sequence
  Description of Artificial Sequence: Synthetic probe
agctctcttg

SEQ ID NO: 10 - DNA, 18 bp, Artificial Sequence
  Description of Artificial Sequence: Synthetic primer
gccgaacgcaccggagca

SEQ ID NO: 11 - DNA, 18 bp, Artificial Sequence
  Description of Artificial Sequence: Synthetic primer
gccgaacgcaccggagta

SEQ ID NO: 12 - DNA, 18 bp, Artificial Sequence
  Description of Artificial Sequence: Synthetic primer
gccgaacgcaccggggca

SEQ ID NO: 13 - DNA, 18 bp, Artificial Sequence
  Description of Artificial Sequence: Synthetic primer
gccgaacgcaccggaaca

SEQ ID NO: 14 - skipped

SEQ ID NO: 15 - DNA, 18 bp, Artificial Sequence
  Description of Artificial Sequence: Synthetic primer
gccgaacgcaccggtgca

SEQ ID NO: 16 - DNA, 23 bp, Artificial Sequence
  Description of Artificial Sequence: Synthetic primer
agcctcttacacccagtggagaa

SEQ ID NO: 17 - DNA, 10 bp, Artificial Sequence
  Description of Artificial Sequence: Synthetic probe
agctctcttg

SEQ ID NO: 18 - DNA, 20 bp, Artificial Sequence
  Description of Artificial Sequence: Synthetic primer
gtgccgaacgcaccggagct

SEQ ID NO: 19 - DNA, 20 bp, Artificial Sequence
  Description of Artificial Sequence: Synthetic primer
  modified_base (17)..(17): t-bb-dA
gtgtcgaacgcaccggagct

SEQ ID NO: 20 - DNA, 17 bp, Artificial Sequence
  Description of Artificial Sequence: Synthetic primer
ccgaacgcaccggagtt

SEQ ID NO: 21 - DNA, 19 bp, Artificial Sequence
  Description of Artificial Sequence: Synthetic primer
tgccgaacgcaccggagtt

SEQ ID NO: 22 - DNA, 20 bp, Artificial Sequence
  Description of Artificial Sequence: Synthetic primer
gtgccgaacgcaccggagtt

SEQ ID NO: 23 - DNA, 19 bp, Artificial Sequence
  Description of Artificial Sequence: Synthetic primer
tgccgaacgcaccggaact

SEQ ID NO: 24 - DNA, 20 bp, Artificial Sequence
  Description of Artificial Sequence: Synthetic primer
gtgccgaacgcaccggaact

SEQ ID NO: 25 - DNA, 23 bp, Artificial Sequence
  Description of Artificial Sequence: Synthetic primer
agcctcttacacccagtggagaa

SEQ ID NO: 26 - DNA, 10 bp, Artificial Sequence
  Description of Artificial Sequence: Synthetic probe
agctctcttg

SEQ ID NO: 27 - DNA, 23 bp, Artificial Sequence
  Description of Artificial Sequence: Synthetic primer
acctccaccgtgcagctcatcat

SEQ ID NO: 28 - DNA, 23 bp, Artificial Sequence
  Description of Artificial Sequence: Synthetic primer
acttccaccgtgcagctcatcct

SEQ ID NO: 29 - DNA, 23 bp, Artificial Sequence
  Description of Artificial Sequence: Synthetic primer
acttccaccgtgcagctcataat

SEQ ID NO: 30 - DNA, 23 bp, Artificial Sequence
  Description of Artificial Sequence: Synthetic primer
tgcgatctgcacacaccagttga

SEQ ID NO: 31 - DNA, 14 bp, Artificial Sequence
  Description of Artificial Sequence: Synthetic probe
agccaatattgtct

SEQ ID NO: 32 - DNA, 22 bp, Artificial Sequence
  Description of Artificial Sequence: Synthetic primer
cagccgaagggcatgagctgca

SEQ ID NO: 33 - DNA, 22 bp, Artificial Sequence
  Description of Artificial Sequence: Synthetic primer
  modified_base (22)..(22): t-bb-dA
cagtcgaagggcatgagctgca

SEQ ID NO: 34 - DNA, 22 bp, Artificial Sequence
  Description of Artificial Sequence: Synthetic primer
  modified_base (21)..(21): t-bb-dC
cagccgaagggcatgagctaca

SEQ ID NO: 35 - DNA, 22 bp, Artificial Sequence
  Description of Artificial Sequence: Synthetic primer
  modified_base (21)..(21): t-bb-dC
cagtcgaagggcatgagctgca

SEQ ID NO: 36 - DNA, 22 bp, Artificial Sequence
  Description of Artificial Sequence: Synthetic primer
  modified_base (21)..(21): t-bb-dC
cagccgaagggcatgagccgca

SEQ ID NO: 37 - DNA, 22 bp, Artificial Sequence
  Description of Artificial Sequence: Synthetic primer
  modified_base (21)..(21): t-bb-dC
cagccgaagggcatgagcagca

SEQ ID NO: 38 - DNA, 22 bp, Artificial Sequence
  Description of Artificial Sequence: Synthetic primer
  modified_base (21)..(21): N4-et-dC
cagccgaagggcatgagccgca

SEQ ID NO: 39 - DNA, 22 bp, Artificial Sequence
  Description of Artificial Sequence: Synthetic primer
  modified_base (21)..(21): N4-et-dC
cagccgaagggcatgagcagca

SEQ ID NO: 40 - DNA, 22 bp, Artificial Sequence
  Description of Artificial Sequence: Synthetic primer
  modified_base (18)..(18): N4-et-dC
  modified_base (21)..(21): t-bb-dC
cagtcgaagggcatgagctgca

SEQ ID NO: 41 - DNA, 24 bp, Artificial Sequence
  Description of Artificial Sequence: Synthetic primer
  modified_base (23)..(23): t-bb-dC
ggcggccgaagggcatgagctgca

SEQ ID NO: 42 - DNA, 24 bp, Artificial Sequence
  Description of Artificial Sequence: Synthetic primer
  modified_base (23)..(23): t-bb-dC
ggcagccgaagggcatgagctaca

SEQ ID NO: 43 - DNA, 24 bp, Artificial Sequence
  Description of Artificial Sequence: Synthetic primer
  modified_base (20)..(20): t-bb-dC
  modified_base (23)..(23): t-bb-dC
ggcggccgaagggcatgagctgca

SEQ ID NO: 44 - DNA, 24 bp, Artificial Sequence
  Description of Artificial Sequence: Synthetic primer
  modified_base (24)..(24): t-bb-dA
ggcggccgaagggcatgagctgca

SEQ ID NO: 45 - DNA, 24 bp, Artificial Sequence
  Description of Artificial Sequence: Synthetic primer
  modified_base (20)..(20): N4-et-dC
  modified_base (24)..(24): t-bb-dA
ggcggccgaagggcatgagctgca

SEQ ID NO: 46 - DNA, 22 bp, Artificial Sequence
  Description of Artificial Sequence: Synthetic primer
cagtcgaagggcatgagcggca

SEQ ID NO: 47 - DNA, 22 bp, Artificial Sequence
  Description of Artificial Sequence: Synthetic primer
cagccgaagggcatgagcggca

SEQ ID NO: 48 - DNA, 24 bp, Artificial Sequence
  Description of Artificial Sequence: Synthetic primer
ggcagccgaagggcatgagcggca

SEQ ID NO: 49 - DNA, 24 bp, Artificial Sequence
  Description of Artificial Sequence: Synthetic primer
cctccctccaggaagcctacgtga

SEQ ID NO: 50 - DNA, 14 bp, Artificial Sequence
  Description of Artificial Sequence: Synthetic probe
tgcacggtggaggt

SEQ ID NO: 51 - DNA, 25 bp, Artificial Sequence
  Description of Artificial Sequence: Synthetic primer
atgtcaagatcacagattttgggcg

SEQ ID NO: 52 - DNA, 25 bp, Artificial Sequence
  Description of Artificial Sequence: Synthetic primer
  modified_base (24)..(24): t-bb-dC
atgttaagatcacagattttgggcg

SEQ ID NO: 53 - DNA, 25 bp, Artificial Sequence
  Description of Artificial Sequence: Synthetic primer
atgtcaagatcacagattttggacg

SEQ ID NO: 54 - DNA, 25 bp, Artificial Sequence
  Description of Artificial Sequence: Synthetic primer
atgtcaagatcacagattttgagcg

SEQ ID NO: 55 - DNA, 25 bp, Artificial Sequence
  Description of Artificial Sequence: Synthetic primer
atgtcaagatcacagattttggggg

SEQ ID NO: 56 - DNA, 25 bp, Artificial Sequence
  Description of Artificial Sequence: Synthetic primer
  modified_base (24)..(24): t-bb-dC
atgtcaagatcacagattttggacg

SEQ ID NO: 57 - DNA, 25 bp, Artificial Sequence
  Description of Artificial Sequence: Synthetic primer
  Description of Combined DNA/RNA Molecule: Synthetic primer
  modified_base (4)..(4): 5-p-dU
  modified_base (10)..(10): 5-p-dU
  modified_base (13)..(13): Me-dC
  modified_base (18)..(18): 5-p-dU
  modified_base (20)..(20): 5-p-dU
  modified_base (24)..(24): t-bb-dC
atgucaagaucacagatutuggacg

SEQ ID NO: 58 - DNA, 22 bp, Artificial Sequence
  Description of Artificial Sequence: Synthetic primer
ctggtccctggtgtcaggaaaa

SEQ ID NO: 59 - DNA, 10 bp, Artificial Sequence
  Description of Artificial Sequence: Synthetic probe
taccatgcag

SEQ ID NO: 60 - DNA, 19 bp, Artificial Sequence
  Description of Artificial Sequence: Synthetic primer
gcacccagcagtttggccc

SEQ ID NO: 61 - DNA, 19 bp, Artificial Sequence
  Description of Artificial Sequence: Synthetic primer
  Description of Combined DNA/RNA Molecule: Synthetic primer
  modified_base (2)..(2): Me-dC
  modified_base (4)..(4): Me-dC
  modified_base (6)..(6): Me-dC
  modified_base (9)..(9): Me-dC
  modified_base (12)..(12): 5-p-dU
  modified_base (14)..(14): 5-p-dU
  modified_base (17)..(17): Me-dC
  modified_base (18)..(18): t-bb-dC
gcgcccagcagutuggccc

SEQ ID NO: 62 - DNA, 19 bp, Artificial Sequence
  Description of Artificial Sequence: Synthetic primer
gcacccagcagtttggctc

SEQ ID NO: 63 - DNA, 19 bp, Artificial Sequence
  Description of Artificial Sequence: Synthetic primer
gcacccagcagtttggcac

SEQ ID NO: 64 - DNA, 19 bp, Artificial Sequence
  Description of Artificial Sequence: Synthetic primer
  modified_base (17)..(17): N4-Et-dC
gcacccagcagtttggcac

SEQ ID NO: 65 - DNA, 19 bp, Artificial Sequence
  Description of Artificial Sequence: Synthetic primer
  modified_base (17)..(17): t-bb-dC
gcacccagcagtttggcac

SEQ ID NO: 66 - DNA, 19 bp, Artificial Sequence
  Description of Artificial Sequence: Synthetic primer
  Description of Combined DNA/RNA Molecule: Synthetic primer
  modified_base (2)..(2): Me-dC
  modified_base (4)..(4): Me-dC
  modified_base (6)..(6): Me-dC
  modified_base (9)..(9): Me-dC
  modified_base (12)..(12): 5-p-dU
  modified_base (14)..(14): 5-p-dU
  modified_base (17)..(17): N4-et-dC
gcacccagcagutuggcac

SEQ ID NO: 67 - DNA, 19 bp, Artificial Sequence
  Description of Artificial Sequence: Synthetic primer
  Description of Combined DNA/RNA Molecule: Synthetic primer
  modified_base (2)..(2): Me-dC
  modified_base (4)..(4): Me-dC
  modified_base (6)..(6): Me-dC
  modified_base (9)..(9): Me-dC
  modified_base (12)..(12): 5-p-dU
  modified_base (14)..(14): 5-p-dU
  modified_base (17)..(17): t-bb-dC
gcacccagcagutuggcac

SEQ ID NO: 68 - DNA, 21 bp, Artificial Sequence
  Description of Artificial Sequence: Synthetic primer
  modified_base (19)..(19): t-bb-dC
ccgcacccagcagtttggcac

SEQ ID NO: 69 - DNA, 22 bp, Artificial Sequence
  Description of Artificial Sequence: Synthetic primer
ctggtccctggtgtcaggaaaa

SEQ ID NO: 70 - DNA, 10 bp, Artificial Sequence
  Description of Artificial Sequence: Synthetic probe
taccatgcag

SEQ ID NO: 71 - DNA, 25 bp, Artificial Sequence
  Description of Artificial Sequence: Synthetic primer
tcacagattttgggctggccaaaca

SEQ ID NO: 72 - DNA, 25 bp, Artificial Sequence
  Description of Artificial Sequence: Synthetic primer
  modified_base (25)..(25): t-bb-dA
tcgcagattttgggctggccaaaca

SEQ ID NO: 73 - DNA, 25 bp, Artificial Sequence
  Description of Artificial Sequence: Synthetic primer
  modified_base (24)..(24): N4-et-dC
tcgcagattttgggctggccaaaca

SEQ ID NO: 74 - DNA, 25 bp, Artificial Sequence
  Description of Artificial Sequence: Synthetic primer
tcgcagattttgggctggccaaata

SEQ ID NO: 75 - DNA, 25 bp, Artificial Sequence
  Description of Artificial Sequence: Synthetic primer
tcgcagattttgggctggccaagca

SEQ ID NO: 76 - DNA, 25 bp, Artificial Sequence
  Description of Artificial Sequence: Synthetic primer
tcgcagattttgggctggccagaca

SEQ ID NO: 77 - DNA, 25 bp, Artificial Sequence
  Description of Artificial Sequence: Synthetic primer
tcgcagattttgggctggccaaaga

SEQ ID NO: 78 - DNA, 25 bp, Artificial Sequence
  Description of Artificial Sequence: Synthetic primer
tcgcagattttgggctggccaatca

SEQ ID NO: 79 - DNA, 25 bp, Artificial Sequence
  Description of Artificial Sequence: Synthetic primer
tcgcagattttgggctggccataca

SEQ ID NO: 80 - DNA, 22 bp, Artificial Sequence
  Description of Artificial Sequence: Synthetic primer
ctggtccctggtgtcaggaaaa

SEQ ID NO: 81 - DNA, 10 bp, Artificial Sequence
  Description of Artificial Sequence: Synthetic probe
taccatgcag

SEQ ID NO: 82 - DNA, 23 bp, Artificial Sequence
  Description of Artificial Sequence: Synthetic primer
ttctttctcttccgcacccagct

SEQ ID NO: 83 - DNA, 23 bp, Artificial Sequence
  Description of Artificial Sequence: Synthetic primer
ttccttctcttccgcacccagct

SEQ ID NO: 84 - DNA, 23 bp, Artificial Sequence
  Description of Artificial Sequence: Synthetic primer
  modified_base (22)..(22): t-bb-dC
ttccttctcttccgcacccagct

SEQ ID NO: 85 - DNA, 23 bp, Artificial Sequence
  Description of Artificial Sequence: Synthetic primer
  modified_base (22)..(22): N4-et-dC
ttccttctcttccgcacccagct

SEQ ID NO: 86 - DNA, 23 bp, Artificial Sequence
  Description of Artificial Sequence: Synthetic primer
ttccttctcttccgcacccagtt

SEQ ID NO: 87 - DNA, 23 bp, Artificial Sequence
  Description of Artificial Sequence: Synthetic primer
ttccttctcttccgcacccaact

SEQ ID NO: 88 - DNA, 23 bp, Artificial Sequence
  Description of Artificial Sequence: Synthetic primer
ttccttctcttccgcacccggct

SEQ ID NO: 89 - DNA, 23 bp, Artificial Sequence
  Description of Artificial Sequence: Synthetic primer
ttccttctcttccgcacccatct

SEQ ID NO: 90 - DNA, 23 bp, Artificial Sequence
  Description of Artificial Sequence: Synthetic primer
ttccttctcttccgcaccctgct

SEQ ID NO: 91 - DNA, 25 bp, Artificial Sequence
  Description of Artificial Sequence: Synthetic primer
gtcttctctgtttcagggcatgaac

SEQ ID NO: 92 - DNA, 10 bp, Artificial Sequence
  Description of Artificial Sequence: Synthetic probe
tactggtgaa

SEQ ID NO: 93 - DNA, 22 bp, Artificial Sequence
  Description of Artificial Sequence: Synthetic primer
aggaagcctacgtgatggccat

SEQ ID NO: 94 - DNA, 22 bp, Artificial Sequence
  Description of Artificial Sequence: Synthetic primer
  modified_base (21)..(21): t-bb-dA
aggaggcctacgtgatggccat

SEQ ID NO: 95 - DNA, 22 bp, Artificial Sequence
  Description of Artificial Sequence: Synthetic primer
  modified_base (20)..(20): t-bb-dC
aggaggcctacgtgatggccat

SEQ ID NO: 96 - DNA, 22 bp, Artificial Sequence
  Description of Artificial Sequence: Synthetic primer
  modified_base (19)..(19): t-bb-dC
aggaggcctacgtgatggccat

SEQ ID NO: 97 - DNA, 22 bp, Artificial Sequence
  Description of Artificial Sequence: Synthetic primer
aggaggcctacgtgatggccgt

SEQ ID NO: 98 - DNA, 22 bp, Artificial Sequence
  Description of Artificial Sequence: Synthetic primer
aggaggcctacgtgatggctat

SEQ ID NO: 99 - DNA, 22 bp, Artificial Sequence
  Description of Artificial Sequence: Synthetic primer
aggaggcctacgtgatggcctt

SEQ ID NO: 100 - DNA, 22 bp, Artificial Sequence
  Description of Artificial Sequence: Synthetic primer
aggaggcctacgtgatggcaat

SEQ ID NO: 101 - DNA, 22 bp, Artificial Sequence
  Description of Artificial Sequence: Synthetic primer
aggaggcctacgtgatggacat

SEQ ID NO: 102 - DNA, 26 bp, Artificial Sequence
  Description of Artificial Sequence: Synthetic primer
ccaatattgtctttgtgttcccggac

SEQ ID NO: 103 - DNA, 14 bp, Artificial Sequence
  Description of Artificial Sequence: Synthetic probe
cacggtggaggtga

SEQ ID NO: 104 - DNA, 19 bp, Artificial Sequence
  Description of Artificial Sequence: Synthetic primer
acgtgggggttgtccacga

SEQ ID NO: 105 - DNA, 19 bp, Artificial Sequence
  Description of Artificial Sequence: Synthetic primer
atgtgggggttgtccacga

SEQ ID NO: 106 - DNA, 19 bp, Artificial Sequence
  Description of Artificial Sequence: Synthetic primer
atgtgggggttgtccgcga

SEQ ID NO: 107 - DNA, 22 bp, Artificial Sequence
  Description of Artificial Sequence: Synthetic primer
atcgcattcatgcgtcttcacc

SEQ ID NO: 108 - DNA, 14 bp, Artificial Sequence
  Description of Artificial Sequence: Synthetic probe
agtgtggcttcgca

SEQ ID NO: 109 - DNA, 23 bp, Artificial Sequence
  Description of Artificial Sequence: Synthetic probe
aggatcttgaaggaaactgaatt

SEQ ID NO: 110 - DNA, 23 bp, Artificial Sequence
  Description of Artificial Sequence: Synthetic probe
aggatcttgaaggaaactgaatt

SEQ ID NO: 111 - DNA, 23 bp, Artificial Sequence
  Description of Artificial Sequence: Synthetic probe
aggatcttgaaggaaactgaatt

SEQ ID NO: 112 - DNA, 15 bp, Artificial Sequence
  Description of Artificial Sequence: Synthetic probe
ttgtgttcccggaca

SEQ ID NO: 113 - DNA, 7 bp, Artificial Sequence
  Description of Artificial Sequence: Synthetic probe
gaggcag

SEQ ID NO: 114 - DNA, 19 bp, Artificial Sequence
  Description of Artificial Sequence: Synthetic probe
aaggaggcaaagtaaggag

SEQ ID NO: 115 - DNA, 19 bp, Artificial Sequence
  Description of Artificial Sequence: Synthetic probe
aaggaggcaaagtaaggag

SEQ ID NO: 116 - DNA, 19 bp, Artificial Sequence
  Description of Artificial Sequence: Synthetic probe
aaggaggcaaagtaaggag

SEQ ID NO: 117 - DNA, 15 bp, Artificial Sequence
  Description of Artificial Sequence: Synthetic probe
aacaccgcagcatgt

SEQ ID NO: 118 - DNA, 9 bp, Artificial Sequence
  Description of Artificial Sequence: Synthetic probe
ggcagatgc

SEQ ID NO: 119 - DNA, 15 bp, Artificial Sequence
  Description of Artificial Sequence: Synthetic probe
tggtggccagaagga

(19) United States
(12) Patent Application Publication
(10) Pub. No.: US 2012/0164641 A1
(43) Pub. Date: Jun. 28, 2012

(54) Methods and Compositions for Detecting Mutation in the Human Epidermal Growth Factor Receptor Gene

(75) Inventors: Keith Bauer, San Rafael, CA (US); Nancy Schoenbrunner, Moraga, CA (US); Alison Tsan, Castro Valley, CA (US)

(73) Assignee: ROCHE MOLECULAR SYSTEMS, INC., Pleasanton, CA (US)

(21) Appl. No.: 13/324,705

(22) Filed: Dec. 13, 2011

Related U.S. Application Data
(60) Provisional application No. 61/426,436, filed on Dec. 22, 2010.

(51) Int. Cl. C12Q 1/68; C07H 21/00

(52) U.S. Cl. 435/6.11; 536/23.1

(57) ABSTRACT

The invention comprises reagents and methods for detecting cancer-causing mutations in the human EGFR gene. Further, a method of detecting the mutations and a method of treatment are disclosed.

### FIELD OF THE INVENTION

The invention relates to cancer diagnostics and companion diagnostics for cancer therapies. In particular, the invention relates to the detection of mutations that are useful for diagnosis and prognosis as well as predicting the effectiveness of treatment of cancer.

### BACKGROUND OF THE INVENTION

Epidermal Growth Factor Receptor (EGFR), also known as HER-1 or Erb-B1, is a member of the type 1 tyrosine kinase family of growth factor receptors. These membrane-bound proteins possess an intracellular tyrosine kinase domain that interacts with various signaling pathways, including the Ras/MAPK, PI3K and AKT pathways. Through these pathways, HER family proteins regulate cell proliferation, differentiation, and survival.

It has been demonstrated that some cancers harbor mutations in the EGFR kinase domain (exons 18-21). (Pao et al. (2004). “EGF receptor gene mutations are common in lung cancers from “never smokers” and are associated with sensitivity of tumors to gefitinib and erlotinib”. P.N.A.S. 101 (36): 13306-13311; Sordella et al. (2004). “Gefitinib-sensitizing EGFR mutations in lung cancer activate anti-apoptotic pathways”. Science 305 (5687): 1163-1167.)

Therapies targeting EGFR have been developed. For example, cetuximab (ERBITUX™) and panitumumab (VECTIBIX™) are anti-EGFR antibodies. Erlotinib (TARCEVA™) and gefitinib (IRESSA™) are quinazolines useful as orally active selective inhibitors of EGFR tyrosine kinase. These drugs are most effective in patients with mutated EGFR gene. For example, Mok et al. (2009) “Gefitinib or carboplatin paclitaxel in pulmonary adenocarcinoma.” N Eng J Med 361:947-957), showed that in patients with EGFR mutation-positive tumors, IRESSA™ prolonged progression-free survival (PFS) compared to chemotherapy. The opposite was true for tumors where EGFR was not mutated: PFS was significantly longer for chemotherapy than IRESSA™. Therefore to improve a patient's chances of successful treatment, EGFR mutations status must be known.

Many mutations in the EGFR gene have been identified in cancer tissues. (U.S. application Ser. No. 12/272,321 filed on Nov. 17, 2008, U.S. Pat. No. 7,294,468). Some mutations in the EGFR kinase domain are common, while others occur less frequently. However, it is essential that a clinical test for EGFR mutations target as many mutations as possible with adequate sensitivity. This will assure that patients with rare mutations do not receive a “false negative” test result and miss out on a potentially life-saving treatment. The challenge is to design an assay that would query for as many cancer-associated EGFR mutations as possible in a cost-effective way.

One technique that is sensitive and amenable to multiplexing is allele-specific PCR (AS-PCR). This technique detects mutations or polymorphisms in nucleic acid sequences in the presence of wild-type variants of the sequences. In a successful allele-specific PCR, the desired variant of the target nucleic acid is amplified, while the other variants are not, at least not to a detectable level. In an allele-specific PCR, at least one primer is allele-specific such that primer extension occurs only when the specific variant of the sequence is present. One or more allele-specific primers targeting one or more polymorphic sites can be present in the same reaction mixture. Design of successful allele-specific primers is an unpredictable art. While it is routine to design a primer for a known sequence, no formula exists for designing a primer that can discriminate between very similar sequences.

In the context of a diagnostic assay, precise discrimination is required. For example, in the context of the EGFR mutation detection, the performance of the allele-specific primer may determine the course of a patient's cancer therapy.

Allele-specific PCR has been applied to the detection of mutations in the EGFR gene. See U.S. application Ser. No. 11/910,511, filed on Apr. 4, 2006. However, there is a need for a comprehensive assay capable of detecting a maximum number of EGFR mutations with maximum specificity and sensitivity.

### SUMMARY OF THE INVENTION

In one embodiment, the invention is an oligonucleotide comprising the primary sequence of oligonucleotides selected from SEQ ID NOs. 2, 10, 18, 27, 32, 51, 60, 71, 82; 93 and 104. In another embodiment, the invention is an oligonucleotide selected from SEQ ID NOs. 3-7, 11-15, 19-24, 28, 29, 33-48, 52-57, 61-68, 72-79, 83-90, 94-101, 105 and 106. In yet another embodiment, the invention is an oligonucleotide selected from SEQ ID NOs. 8, 16, 25, 30, 49, 58, 69, 80, 91, 102 and 107. In yet another embodiment, the invention is an oligonucleotide selected from SEQ ID NOs. 9, 17, 26, 31, 50, 59, 70, 81, 92, 103 and 108.

In a further embodiment, the invention is a method of detecting mutations in the human epidermal growth factor receptor (EGFR) nucleic acid in a sample comprising: contacting the nucleic acid in the sample with the oligonucleotide of claim 1; incubating the sample under conditions allowing hybridization of the oligonucleotide to the target sequence within the EGFR nucleic acid; generation of the amplification product containing the target sequence within the EGFR nucleic acid; and detecting the presence of the amplified product thereby detecting the presence of the mutation in the EGFR nucleic acid.

In a further embodiment, the invention is a method of treating a patient having a tumor possibly harboring cells with a mutation in the epidermal growth factor receptor (EGFR) gene, comprising: contacting the nucleic acid in the sample from the patient with the oligonucleotide of claim 1; incubating the sample under conditions allowing hybridization of the oligonucleotide to the target sequence within the EGFR nucleic acid; generation of the amplification product containing the target sequence within the EGFR nucleic acid; detecting the presence of the amplified product thereby detecting the presence of the mutation in the EGFR nucleic acid, and if a mutation is present, administering to the patient a compound that inhibits signaling of the mutant EGFR protein encoded by the mutated gene.

In a yet further embodiment, the invention is a method of determining whether a treatment of a patient with a malignant tumor with EGFR inhibitors is likely to be successful, comprising: contacting the nucleic acid in the sample from the patient with the oligonucleotide of claim 1; incubating the sample under conditions allowing hybridization of the oligonucleotide to the target sequence within the EGFR nucleic acid; generation of the amplification product containing the target sequence within the EGFR nucleic acid; detecting the presence of the amplified product thereby detecting the presence of the mutation in the EGFR nucleic acid, and if a mutation is present, determining that the treatment is likely to be successful.

In a further embodiment, the invention is a kit comprising one or more pairs of oligonucleotides selected from pairs (a)-(k): (a) an oligonucleotide of one of SEQ ID NOs: 2-7 and the oligonucleotide of SEQ ID NO: 8; (b) an oligonucleotide of one of SEQ ID NOs: 10-15 and the oligonucleotide of SEQ ID NO: 16; (c) an oligonucleotide of one of SEQ ID NOs: 18-24 and the oligonucleotide of SEQ ID NO: 25; (d) an oligonucleotide of one of SEQ ID NOs: 27-29 and the oligonucleotide of SEQ ID NO: 30; (e) an oligonucleotide of one of SEQ ID NOs: 32-48 and the oligonucleotide of SEQ ID NO: 49; (f) an oligonucleotide of one of SEQ ID NOs: 51-57 and the oligonucleotide of SEQ ID NO: 58; (g) an oligonucleotide of one of SEQ ID NOs: 60-68 and the oligonucleotide of SEQ ID NO: 69; (h) an oligonucleotide of one of SEQ ID NOs: 71-79 and the oligonucleotide of SEQ ID NO: 80; (i) an oligonucleotide of one of SEQ ID NOs: 82-90 and the oligonucleotide of SEQ ID NO: 91; (j) an oligonucleotide of one of SEQ ID NOs: 93-101 and the oligonucleotide of SEQ ID NO: 102; (k) an oligonucleotide of one of SEQ ID NOs: 104-106 and the oligonucleotide of SEQ ID NO: 107.

In a yet further embodiment, in the invention is a reaction mixture for detecting mutations in the human epidermal growth factor receptor (EGFR) gene comprising one or more pairs of oligonucleotides selected from pairs (a)-(k): an oligonucleotide of one of SEQ ID NOs: 2-7 and the oligonucleotide of SEQ ID NO: 8; an oligonucleotide of one of SEQ ID NOs: 10-15 and the oligonucleotide of SEQ ID NO: 16; an oligonucleotide of one of SEQ ID NOs: 18-24 and the oligonucleotide of SEQ ID NO: 25; an oligonucleotide of one of SEQ ID NOs: 27-29 and the oligonucleotide of SEQ ID NO: 30; an oligonucleotide of one of SEQ ID NOs: 32-48 and the oligonucleotide of SEQ ID NO: 49; an oligonucleotide of one of SEQ ID NOs: 51-57 and the oligonucleotide of SEQ ID NO: 58; an oligonucleotide of one of SEQ ID NOs: 60-68 and the oligonucleotide of SEQ ID NO: 69; an oligonucleotide of one of SEQ ID NOs: 71-79 and the oligonucleotide of SEQ ID NO: 80; an oligonucleotide of one of SEQ ID NOs: 82-90 and the oligonucleotide of SEQ ID NO: 91; an oligonucleotide of one of SEQ ID NOs: 93-101 and the oligonucleotide of SEQ ID NO: 102; an oligonucleotide of one of SEQ ID NOs: 104-106 and the oligonucleotide of SEQ ID NO: 107.

### BRIEF DESCRIPTION OF THE DRAWINGS

FIG. 1(A-C) shows the coding sequence of the human EGFR gene (SEQ ID NO: 1).

### DETAILED DESCRIPTION OF THE INVENTION

### Definitions

To facilitate the understanding of this disclosure, the following definitions of the terms used herein are provided.

The term “X[n]Y” refers to a missense mutation that results in a substitution of amionacid X for amino acid Y at position [n] within the amino acid sequence. For example, the term “G719A” refers to a mutation where glycine at position 719 is replaced with alanine.

The term “allele-specific primer” or “AS primer” refers to a primer that hybridizes to more than one variant of the target sequence, but is capable of discriminating between the variants of the target sequence in that only with one of the variants, the primer is efficiently extended by the nucleic acid polymerase under suitable conditions. With other variants of the target sequence, the extension is less efficient or inefficient.

The term “common primer” refers to the second primer in the pair of primers that includes an allele-specific primer. The common primer is not allele-specific, i.e. does not discriminate between the variants of the target sequence between which the allele-specific primer discriminates.

The terms “complementary” or “complementarity” are used in reference to antiparallel strands of polynucleotides related by the Watson-Crick base-pairing rules. The terms “perfectly complementary” or “100% complementary” refer to complementary sequences that have Watson-Crick pairing of all the bases between the antiparallel strands, i.e. there are no mismatches between any two bases in the polynucleotide duplex. However, duplexes are formed between antiparallel strands even in the absence of perfect complementarity. The terms “partially complementary” or “incompletely complementary” refer to any alignment of bases between antiparallel polynucleotide strands that is less than 100% perfect (e.g., there exists at least one mismatch or unmatched base in the polynucleotide duplex). The duplexes between partially complementary strands are generally less stable than the duplexes between perfectly complementary strands.

The term “sample” refers to any composition containing or presumed to contain nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom.

The terms “polynucleotide” and “oligonucleotide” are used interchangeably. “Oligonucleotide” is a term sometimes used to describe a shorter polynucleotide. An oligonucleotide may be comprised of at least 6 nucleotides, for example at least about 10-12 nucleotides, or at least about 15-30 nucleotides corresponding to a region of the designated nucleotide sequence.

The term “primary sequence” refers to the sequence of nucleotides in a polynucleotide or oligonucleotide. Nucleotide modifications such as nitrogenous base modifications, sugar modifications or other backbone modifications, are not a part of the primary sequence. Labels, such as chromophores conjugated to the oligonucleotides are also not a part of the primary sequence. Thus two oligonucleotides can share the same primary sequence but differ with respect to the modifications and labels.

The term “primer” refers to an oligonucleotide which hybridizes with a sequence in the target nucleic acid and is capable of acting as a point of initiation of synthesis along a complementary strand of nucleic acid under conditions suitable for such synthesis. As used herein, the term “probe” refers to an oligonucleotide which hybridizes with a sequence in the target nucleic acid and is usually detectably labeled. The probe can have modifications, such as a 3′-terminus modification that makes the probe non-extendable by nucleic acid polymerases, and one or more chromophores. An oligonucleotide with the same sequence may serve as a primer in one assay and a probe in a different assay.

As used herein, the term “target sequence”, “target nucleic acid” or “target” refers to a portion of the nucleic acid sequence which is to be either amplified, detected or both.

The terms “hybridized” and “hybridization” refer to the base-pairing interaction of between two nucleic acids that results in formation of a duplex. It is not a requirement that two nucleic acids have 100% complementarity over their full length to achieve hybridization.

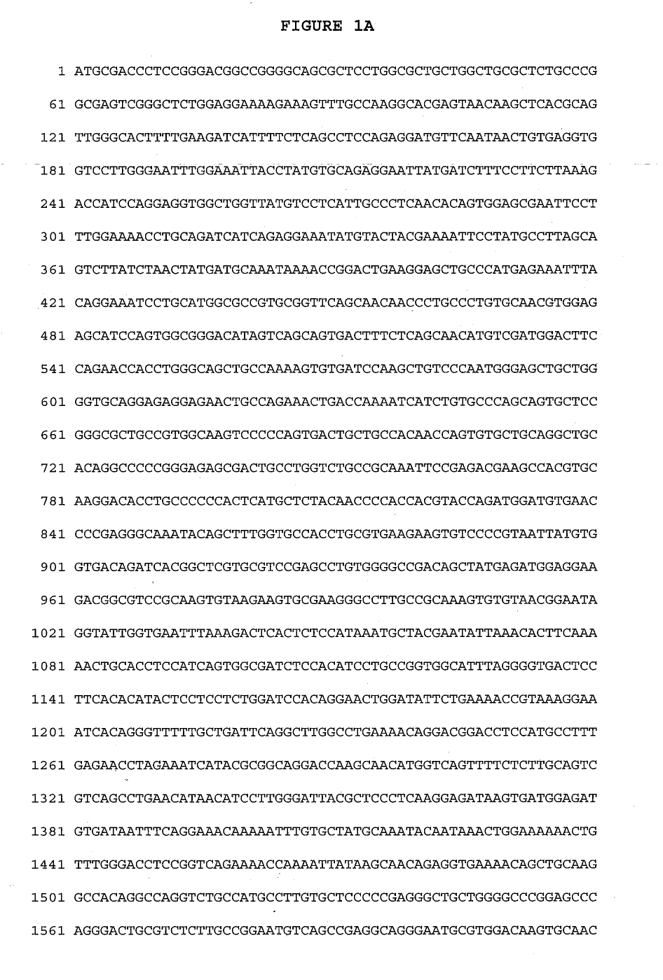

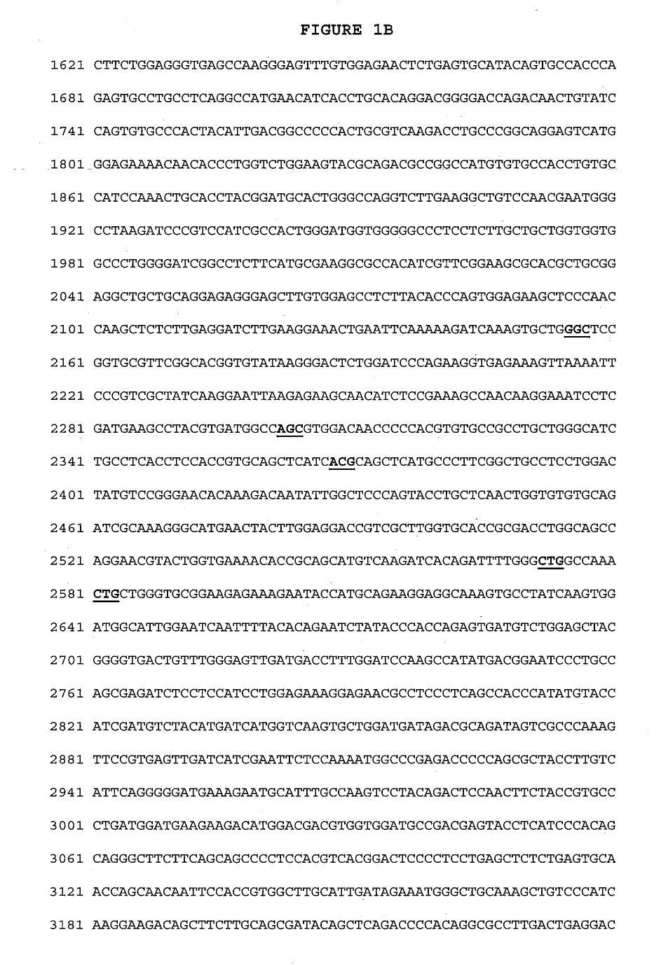

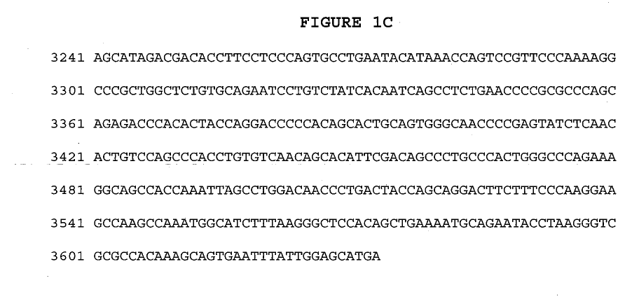

The coding portion of the human EGFR cDNA (SEQ ID No: 1) is shown on FIG. 1 (1A-1C) (Ensembl ref. no. ENST00000275493, www.ensembl.org, see Hubbard et al. (2009) Ensembl 2009, Nucl. Acids Res. 37 (suppl 1): D690-D697)), which is a portion of the complete EGFR cDNA sequence (NCBI accession No. NM_{—}005228.3). Some of the codons frequently mutated in cancer patients are underlined and shown in bold.

Allele-specific PCR has been described in U.S. Pat. No. 6,627,402. In an allele-specific PCR, the discriminating primer has a sequence complementary to the desired variant of the target sequence, but mismatched with the undesired variants of the target sequence. Typically, the discriminating nucleotide in the primer, i.e. the nucleotide matching only one variant of the target sequence, is the 3′-terminal nucleotide. However, the 3′ terminus of the primer is only one of many determinants of specificity. The specificity in an allele-specific PCR derives from the much slower rate of extension of the mismatched primer than of the matched primer, ultimately reducing the relative amplification efficiency of the mismatched target. The reduced extension kinetics and thus PCR specificity is influenced by many factors including the nature of the enzyme, reaction components and their concentrations, the extension temperature and the overall sequence context of the mismatch. The effect of these factors on each particular primer cannot be reliably quantified. Without a reliable quantitative strategy and with an enormous number of variables, the design of allele-specific primers is a matter of trial and error with often surprising results. In the case of mutant alleles of EGFR described below, only a fraction of primers tested gave suitable performance, i.e. acceptable PCR efficiency and at the same time, discrimination between the mutant and the wild-type template.

One approach to increasing specificity of allele-specific primers is by including an internal mismatched nucleotide in addition to the terminal mismatch. See U.S. patent application Ser. No. 12/582,068 filed on Oct. 20, 2009, which is incorporated herein by reference in its entirety. The internal mismatched nucleotide in the primer may be mismatched with both the desired and the undesired target sequences. Because the mismatches destabilize the primer-template hybrids with both desired and undesired templates, some of the mismatches can prevent amplification of both templates and cause failure of the PCR. Therefore the effect of these internal mismatches on a particular allele-specific PCR primer cannot be predicted.

For successful extension of a primer, the primer needs to have at least partial complementarity to the target sequence. Generally, complementarity at the 3′-end of the primer is more critical than complementarity at the 5′-end of the primer. (Innis et al. Eds. PCR Protocols, (1990) Academic Press, Chapter 1, pp. 9-11). Therefore the present invention encompasses the primers disclosed in Tables 1-7 as well as the variants of these primers with 5′-end variations.

It has been previously described that for PCR amplification in general, primer specificity can be increased by the use of chemical modification of the nucleotides in the primer. The nucleotides with covalent modifications of the exocyclic amino groups and the use of such nucleotides in PCR have been described in U.S. Pat. No. 6,001,611, which is incorporated herein by reference in its entirety. Because the modifications disrupt Watson-Crick hydrogen bonding in primer-template hybrids with both desired and undesired templates, some of the modifications can prevent amplification of both templates and cause failure of the PCR. Therefore the effect of these covalent modifications on allele-specific PCR cannot be predicted.

In one embodiment the present invention comprises oligonucleotides for simultaneously detecting multiple EGFR mutations in a single tube. In one embodiment, the invention comprises oligonucleotides (SEQ ID NOS: 2-108) for specifically detecting mutations in the human EGFR gene (Tables 1-7). Some of these primers contain internal mismatches and covalent modifications as shown in Tables 1-7. As an option, the allele-specific primers of the present invention may be paired with a “common” i.e. not allele-specific second primer. The use of the disclosed second primer is optional. Any other suitable downstream primer can be paired with the allele-specific primers of the present invention.

In another embodiment, the present invention is a diagnostic method of detecting EGFR mutations using the primers disclosed in Tables 1-7. The method comprises contacting a test sample of nucleic acid with one or more allele-specific primer for a EGFR mutation selected from Tables 1-7 in the presence of the corresponding second primer, (optionally, also selected from Tables 1-7), nucleoside triphosphates and a nucleic acid polymerase, such that the one or more allele-specific primers is efficiently extended only when an EGFR mutation is present in the sample; and detecting the presence or absence of an EGFR mutation by detecting the presence or absence of the extension product.

In a particular embodiment the presence of the extension product is detected with a probe. In variations of this embodiment the probe is selected from Tables 1-7. The probe may be labeled with a radioactive, a fluorescent or a chromophore label. For example, the mutation may be detected by detecting amplification of the extension product by real-time polymerase chain reaction (rt-PCR), where hybridization of the probe to the extension product results in enzymatic digestion of the probe and detection of the resulting fluorescence (TaqMan™ probe method, Holland et al. (1991) P.N.A.S. USA 88:7276-7280). The presence of the amplification product in rt-PCR may also be detected by detecting a change in fluorescence due to the formation of a nucleic acid duplex between the probe and the extension product. (U.S. application Ser. No. 12/330,694, filed on Dec. 9, 2008). Alternatively, the presence of the extension product and the amplification product may be detected by gel electrophoresis followed by staining or by blotting and hybridization as described e.g., in Sambrook, J. and Russell, D. W. (2001) Molecular Cloning, 3rd ed. CSHL Press, Chapters 5 and 9.

In another embodiment, the invention is a method of treating a patient having a tumor possibly harboring cells with a mutant EGFR gene. The method comprises contacting a sample from the patient with one or more allele-specific primers for a EGFR mutation selected from Tables 1-7 in the presence of a corresponding second primer or primers, (optionally, also selected from Tables 1-7), conducting allele-specific amplification, and detecting the presence or absence of an EGFR mutation by detecting presence or absence of the extension product, and if at least one mutation is found, administering to the patient a compound that inhibits signaling of the mutant EGFR protein encoded by the mutated gene. For each mutation, detection may be performed using a corresponding probe (optionally, also selected from Tables 1-7).

In another embodiment, the invention is a method of determining whether a treatment of a patient with a malignant tumor with EGFR inhibitors is likely to be successful. The method comprises contacting a sample from the patient with one or more allele-specific primers for a EGFR mutation selected from Tables 1-7 in the presence of one or more corresponding second primers, (optionally, also selected from Tables 1-7), conducting allele-specific amplification, and detecting the presence or absence of an EGFR mutation by detecting presence or absence of the extension product, and if at least one mutation is found, determining that the treatment is likely to be successful. For each mutation, detection may be performed using a corresponding probe (optionally, also selected from Tables 1-7). In variations of this embodiment, the EGFR inhibitors are cetuximab, panitumumab, erlotinib and gefitinib.

In yet another embodiment, the invention is a kit containing reagents necessary for detecting mutations in the EGFR gene. The reagents comprise one or more allele-specific primers for an EGFR mutation selected from Tables 1-7, one or more corresponding second primers (optionally also selected from Tables 1-7), and optionally, one or more probes (optionally also selected from Tables 1-7). The kit may further comprise reagents necessary for the performance of amplification and detection assay, such as nucleoside triphosphates, nucleic acid polymerase and buffers necessary for the function of the polymerase. In some embodiments, the probe is detectably labeled. In such embodiments, the kit may comprise reagents for labeling and detecting the label.

In yet another embodiment, the invention is a reaction mixture for detecting mutations in the EGFR gene. The mixture comprises one or more allele-specific primers for an EGFR mutation selected from Tables 1-7, one or more corresponding second primers (optionally also selected from Tables 1-7), and optionally, one or more probes (optionally also selected from Tables 1-7). The reaction mixture may further comprise reagents such as nucleoside triphosphates, nucleic acid polymerase and buffers necessary for the function of the polymerase.

### EXAMPLES

### Exemplary Reaction Conditions

The exemplary reaction conditions used for testing the performance of the primers are as follows. A PCR mixture including 50 mM Tris-HCl (pH 8.0), 80-100 mM potassium chloride, 200 μM each dATP, dCTP and dGTP, 400 μM dUTP, 0.1 μM each of selective and common primer, 0.05 μM probe, target DNA (10,000 copies of a plasmid with a mutant, or 10,000 copies of wild-type genomic DNA (pooled genomic DNA, Clontech, Mountain View, Calif., Cat. No. 636401), 0.02 U/uL uracil-N-glycosylase, 200 nM NTQ21-46A aptamer, 20 nM DNA polymerase, 0.1 mM EDTA, 2.6 mM magnesium acetate. Amplification and analysis was done using the Roche LightCycler® 480 instrument (Roche Applied Science, Indianapolis, Ind.) The following temperature profile was used: 95° C. for 1 minute (or 2 cycles of 95° C. (10 seconds) to 62° C. (25 seconds) followed by cycling from 92° C. (10 seconds) to 62° C. (25-30 seconds) 99 times. Fluorescence data was collected at the start of each 62° C. step. Optionally, the reactions contained an endogenous positive control template.

Discrimination between the wild-type and mutant sequences was measured as the difference between the cycles-to-threshold (ΔCₜ) values for the wild-type and mutant targets. For example, AG of 29 cycles was recorded when a reaction with the mutant target reached the threshold cycle after 26 cycles, and the reaction with the wild-type target reached the threshold cycle only after 55 cycles.

Legends to the Tables

The following abbreviations are used for the modified-base nucleotides: “t-bb-dA” and “t-bb-dC” mean N6-tert-butyl-benzyl-deoxyadenine and N4-tert-butyl-benzyl-deoxycytosine respectively; the term “et-dC” means N4-ethyl-deoxycytosine; the term “met-dC” means N4-methyl-deoxycytosine; and the term “5-p-dU” means 5-propynyl-deoxyuracil. In the primer and probe sequences, the bold, underlined nucleotides are modified-base nucleotides, or nucleotides mismatched with both the wild-type and the mutant sequence.

### Example 1

### Primers for Detecting Mutation G719A in the Human EGFR Gene

This mutation results from the nucleotide change 2156 G->C in the EGFR gene (SEQ ID NO: 1). Primers and probes for detecting the mutation are shown in Table 1. The mutation may be detected using an allele-specific primer selected from SEQ ID NOs: 2-7 and a common primer. Optionally, the common primer may be SEQ ID NO: 8. The amplification may be detected using a probe that hybridizes to the region between the allele-specific and a common primer. Optionally, the probe may be SEQ ID NO: 9.

TABLE 1
Primers and probes for detecting mutation G719A

| | | | |
|---|---|---|---|
| SEQ ID NO: 2 | AS primer | GCCGAACGCACCGGAGG | |
| SEQ ID NO: 3 | AS primer | GCCGAACGCACCGGGGG | |
| SEQ ID NO: 4 | AS primer | GCCGAACGCACCGAAGG | |
| SEQ ID NO: 5 | AS primer | GCCGAACGCACCGGTGG | |
| SEQ ID NO: 6 | AS primer | GCCGAACGCACCGCAGG | |
| SEQ ID NO: 7 | AS primer | GTGTCGAACGCACCGGEGG | E = t-bb-dA |
| SEQ ID NO: 8 | Common | AGCCTCTTACACCCAGTGGAGAA | |
| | primer | | |
| SEQ ID NO: 9 | probe | HAGCTCTCTTGQAGGATCTTGAAGGAAACTGAATTP | |

H = Hex, Q = BHQ-2, P = phosphate

The allele-specific primers disclosed in this example achieved discrimination between the wild-type sequence and the G719A mutation of ΔCₜ up to 68 cycles, depending on reaction conditions.

### Example 2

### Primers for Detecting the Mutation G719C in the Human EGFR Gene

This mutation results from the nucleotide change 2156 G->T in the EGFR gene (SEQ ID NO: 1). Primers and probes for detecting the mutation are shown in Table 2. The mutation may be detected using an allele-specific primer selected from SEQ ID NOs: 10-15 and a common primer. Optionally, the common primer may be SEQ ID NO: 16. The amplification may be detected using a probe that hybridizes to the region between the allele-specific and a common primer. Optionally, the probe may be SEQ ID NO: 17.

TABLE 2
Primers and probes for detecting mutation G719C

| | | |
|---|---|---|
| SEQ ID NO: 10 | AS primer | GCCGAACGCACCGGAGCA |
| SEQ ID NO: 11 | AS primer | GCCGAACGCACCGGAGTA |
| SEQ ID NO: 12 | AS primer | GCCGAACGCACCGGGGCA |
| SEQ ID NO: 13 | AS primer | GCCGAACGCACCGGAACA |
| SEQ ID NO: 15 | AS primer | GCCGAACGCACCGGTGCA |
| SEQ ID NO: 16 | Common primer | AGCCTCTTACACCCAGTGGAGAA |
| SEQ ID NO: 17 | probe | HAGCTCTCTTGQAGGATCTTGAAGGAAACTGAATTP |

H = Hex, Q = BHQ-2, P = phosphate

The allele-specific primers disclosed in this example achieved discrimination between the wild-type sequence and the G719C mutation of ΔCₜ up to 69 cycles, depending on reaction conditions.

### Example 3

### Primers for Detecting Mutation G719S in the Human EGFR Gene

This mutation results from the nucleotide change 2155-2156 GG->TC in the EGFR gene (SEQ ID NO: 1). Primers and probes for detecting the mutation are shown in Table 3. The mutation may be detected using an allele-specific primer selected from SEQ ID NOs: 18-24 and a common primer. Optionally, the common primer may be SEQ ID NO: 25. The amplification may be detected using a probe that hybridizes to the region between the allele-specific and a common primer. Optionally, the probe may be SEQ ID NO: 26.

TABLE 3
Primers and probes for detecting mutation G719S

| | | | |
|---|---|---|---|
| SEQ ID NO: 18 | AS | GTGCCGAACGCACCGGAGCT | |
| | primer | | |
| SEQ ID NO: 19 | AS | GTGTCGAACGCACCGGEGCT | E = t-bb-dA |
| | primer | | |
| SEQ ID NO: 20 | AS | CCGAACGCACCGGAGTT | |
| | primer | | |
| SEQ ID NO: 21 | AS | TGCCGAACGCACCGGAGTT | |
| | primer | | |
| SEQ ID NO: 22 | AS | GTGCCGAACGCACCGGAGTT | |
| | primer | | |
| SEQ ID NO: 23 | AS | TGCCGAACGCACCGGAACT | |
| | primer | | |
| SEQ ID NO: 24 | AS | GTGCCGAACGCACCGGAACT | |
| | primer | | |
| SEQ ID NO: 25 | Common | AGCCTCTTACACCCAGTGGAGAA | |
| | primer | | |
| SEQ ID NO: 26 | probe | HAGCTCTCTTGQAGGATCTTGAAGGAAACTGAATTP | |

H = Hex, Q = BHQ-2, P = phosphate

### Example 4

### Primers for Detecting Mutation T790M in the Human EGFR Gene

This mutation results from the nucleotide change 2369 C->T in the EGFR gene (SEQ ID NO: 1). Primers and probes for detecting the mutation are shown in Tables 4a and 4b. The mutation may be detected using an allele-specific primer selected from SEQ ID NOs: 27-29 and a common primer. Optionally, the common primer may be SEQ ID NO: 30. The amplification may be detected using a probe that hybridizes to the region between the allele-specific and a common primer. Optionally, the probe may be SEQ ID NO: 31. If the antisense strand is used, the mutation may be detected using an allele-specific primer selected from SEQ ID NOs: 32-48 and a common primer. Optionally, the common primer may be SEQ ID NO: 49. The amplification may be detected using a probe that hybridizes to the region between the allele-specific and a common primer. Optionally, the probe may b& SEQ ID NO: 50.

TABLE 4a
Primers and probes for detecting mutation T790M (sense)

| | | |
|---|---|---|
| SEQ ID NO: 27 | AS primer | ACCTCCACCGTGCAGCTCATCAT |
| SEQ ID NO: 28 | AS primer | ACTTCCACCGTGCAGCTCATCCT |
| SEQ ID NO: 29 | AS primer | ACTTCCACCGTGCAGCTCATAAT |
| SEQ ID NO: 30 | Common primer | TGCGATCTGCACACACCAGTTGA |
| SEQ ID NO: 31 | probe | JAGCCAATATTGTCTQTTGTGTTCCCGGACAP |

J = Ja270, Q = BHQ-2, P = phosphate

TABLE 4b
Primers and probes for detecting mutation T790M (antisense)

| | | | |
|---|---|---|---|
| SEQ ID NO: 32 | AS primer | CAGCCGAAGGGCATGAGCTGCA | |
| SEQ ID NO: 33 | AS primer | CAGTCGAAGGGCATGAGCTGCE | E = t-bb-dA |
| SEQ ID NO: 34 | AS primer | CAGCCGAAGGGCATGAGCTAEA | E = t-bb-dC |
| SEQ ID NO: 35 | AS primer | CAGTCGAAGGGCATGAGCTGEA | E = t-bb-dC |
| SEQ ID NO: 36 | AS primer | CAGCCGAAGGGCATGAGCCGEA | E = t-bb-dC |
| SEQ ID NO: 37 | AS primer | CAGCCGAAGGGCATGAGCAGEA | E = t-bb-dC |
| SEQ ID NO: 38 | AS primer | CAGCCGAAGGGCATGAGCCGJA | J = N4-et-dC |
| SEQ ID NO: 39 | AS primer | CAGCCGAAGGGCATGAGCAGJA | J = N4-et-dC |
| SEQ ID NO: 40 | AS primer | CAGTCGAAGGGCATGAGJTGEA | E = t-bb-dC |
| | | | J = N4-et-dC |
| SEQ ID NO: 41 | AS primer | GGCGGCCGAAGGGCATGAGCTGEA | E = t-bb-dC |
| SEQ ID NO: 42 | AS primer | GGCAGCCGAAGGGCATGAGCTAEA | E = t-bb-dC |
| SEQ ID NO: 43 | AS primer | GGCGGCCGAAGGGCATGAGETGEA | E = t-bb-dC |
| SEQ ID NO: 44 | AS primer | GGCGGCCGAAGGGCATGAGCTGCE | E = t-bb-dA |
| SEQ ID NO: 45 | AS primer | GGCGGCCGAAGGGCATGAGJTGCE | E = t-bb-dA |
| | | | J = N4-et-dC |
| SEQ ID NO: 46 | AS primer | CAGTCGAAGGGCATGAGCGGCA | |
| SEQ ID NO: 47 | AS primer | CAGCCGAAGGGCATGAGCGGCA | |
| SEQ ID NO: 48 | AS primer | GGCAGCCGAAGGGCATGAGCGGCA | |
| SEQ ID NO: 49 | Common | CCTCCCTCCAGGAAGCCTACGTGA | |
| | primer | | |
| SEQ ID NO: 50 | probe | JTGCACGGTGGAGGTQGAGGCAGP | |

J = Ja270, Q = BHQ-2, P = phosphate

The allele-specific primers disclosed in this example achieved discrimination between the wild-type sequence and the T790M mutation of ΔCₜ up to 51 cycles, depending on reaction conditions.

### Example 5

### Primers for Detecting Mutation L858R in the Human EGFR Gene

This mutation results from the nucleotide change 2573 T->G in the EGFR gene (SEQ ID NO: 1). Primers and probes for detecting the mutation are shown in Table 5. The mutation may be detected using an allele-specific primer selected from SEQ ID NOs: 51-57 and a common primer. Optionally, the common primer may be SEQ ID NO: 58. The amplification may be detected using a probe that hybridizes to the region between the allele-specific and a common primer. Optionally, the probe may be SEQ ID NO: 59. If the antisense strand is used, the mutation may be detected using an allele-specific primer selected from SEQ ID NOs: 60-68 and a common primer. Optionally, the common primer may be SEQ ID NO: 69. The amplification may be detected using a probe that hybridizes to the region between the allele-specific and a common primer. Optionally, the probe may be SEQ ID NO: 70.

TABLE 5
Primers and probes for detecting mutation L858R

| | | | |
|---|---|---|---|
| SEQ ID NO: 51 | AS primer | ATGTCAAGATCACAGATTTTGGGCG | |
| SEQ ID NO: 52 | AS primer | ATGTTAAGATCACAGATTTTGGGJG | J = t-bb-dC |
| SEQ ID NO: 53 | AS primer | ATGTCAAGATCACAGATTTTGGACG | |
| SEQ ID NO: 54 | AS primer | ATGTCAAGATCACAGATTTTGAGCG | |
| SEQ ID NO: 55 | AS primer | ATGTCAAGATCACAGATTTTGGGGG | |
| SEQ ID NO: 56 | AS primer | ATGTCAAGATCACAGATTTTGGAJG | J = t-bb-dC |
| SEQ ID NO: 57 | AS primer | ATGUCAAGAUCAEAGATUTUGGAJG | E = Me-dC |
| | | | J = t-bb-dC |
| | | | U = 5-p-dU |
| SEQ ID NO: 58 | Common | CTGGTCCCTGGTGTCAGGAAAA | |
| | primer | | |
| SEQ ID NO: 59 | probe | FTACCATGCAGQAAGGAGGCAAAGTAAGGAGP | |
| SEQ ID NO: 60 | AS primer | GCACCCAGCAGTTTGGCCC | |
| SEQ-ID-NO: 61 | AS primer | GEGECEAGEAGUTUGGEJC | E = Me-dC |
| | | | J = t-bb-dC |
| | | | U = 5-p-dU |
| SEQ ID NO: 62 | AS primer | GCACCCAGCAGTTTGGCTC | |
| SEQ ID NO: 63 | AS primer | GCACCCAGCAGTTTGGCAC | |
| SEQ ID NO: 64 | AS primer | GCACCCAGCAGTTTGGJAC | J = N4-Et-dC |
| SEQ ID NO: 65 | AS primer | GCACCCAGCAGTTTGGJAC | J = t-bb-dC |
| SEQ ID NO: 66 | AS primer | GEAECEAGEAGUTUGGJAC | E = Me-dC |
| | | | J = N4-et-dC, |
| | | | U = 5-p-dU |
| SEQ ID NO: 67 | AS primer | GEAECEAGEAGUTUGGJAC | E = Me-dC |
| | | | J = t-bb-dC |
| | | | U = 5-p-dU |
| SEQ ID NO: 68 | AS primer | CCGCACCCAGCAGTTTGGJAC | J = t-bb-dC |
| SEQ ID NO: 69 | Common | CTGGTCCCTGGTGTCAGGAAAA | |
| | primer | | |
| SEQ ID NO: 70 | probe | FTACCATGCAGQAAGGAGGCAAAGTAAGGAGP | |

F = FAM, Q = BHQ-2, P = phosphate

The allele-specific primers disclosed in this example achieved discrimination between the wild-type sequence and the L858R mutation of ΔCₜ up to 69 cycles, depending on reaction conditions.

### Example 6

### Primers for Detecting Mutation L861 Q in the Human EGFR Gene

This mutation results from the nucleotide change 2582 T->A in the EGFR gene (SEQ ID NO: 1). Primers and probes for detecting the mutation are shown in Table 6. The mutation may be detected using an allele-specific primer selected from SEQ ID NOs: 71-79 and a common primer. Optionally, the common primer may be SEQ ID NO: 80. The amplification may be detected using a probe that hybridizes to the region between the allele-specific and a common primer. Optionally, the probe may be SEQ ID NO: 81. If the antisense strand is used, the mutation may be detected using an allele-specific primer selected from SEQ ID NOs: 82-90 and a common primer. Optionally, the common primer may be SEQ ID NO: 91. The amplification may be detected using a probe that hybridizes to the region between the allele-specific and a common primer. Optionally, the probe may be SEQ ID NO: 92.

TABLE 6
Primers and probes for detecting mutation L861Q

| | | | |
|---|---|---|---|
| SEQ ID NO: 71 | AS primer | TCACAGATTTTGGGCTGGCCAAACA | |
| SEQ ID NO: 72 | AS primer | TCGCAGATTTTGGGCTGGCCAAACE | E = t-bb-dA |
| SEQ ID NO: 73 | AS primer | TCGCAGATTTTGGGCTGGCCAAAEA | E = N4-et-dC |
| SEQ ID NO: 74 | AS primer | TCGCAGATTTTGGGCTGGCCAAATA | |
| SEQ ID NO: 75 | AS primer | TCGCAGATTTTGGGCTGGCCAAGCA | |
| SEQ ID NO: 76 | AS primer | TCGCAGATTTTGGGCTGGCCAGACA | |
| SEQ ID NO: 77 | AS primer | TCGCAGATTTTGGGCTGGCCAAAGA | |
| SEQ ID NO: 78 | AS primer | TCGCAGATTTTGGGCTGGCCAATCA | |
| SEQ ID NO: 79 | AS primer | TCGCAGATTTTGGGCTGGCCATACA | |
| SEQ ID NO: 80 | Common | CTGGTCCCTGGTGTCAGGAAAA | |
| | primer | | |
| SEQ ID NO: 81 | probe | FTACCATGCAGQAAGGAGGCAAAGTAAGGAGP | |
| SEQ ID NO: 82 | AS primer | TTCTTTCTCTTCCGCACCCAGCT | |
| SEQ ID NO: 83 | AS primer | TTCCTTCTCTTCCGCACCCAGCT | |
| SEQ ID NO: 84 | AS primer | TTCCTTCTCTTCCGCACCCAGET | E = t-bb-dC |
| SEQ ID NO: 85 | AS primer | TTCCTTCTCTTCCGCACCCAGET | E = N4-et-dC |
| SEQ ID NO: 86 | AS primer | TTCCTTCTCTTCCGCACCCAGTT | |
| SEQ ID NO: 87 | AS primer | TTCCTTCTCTTCCGCACCCAACT | |
| SEQ ID NO: 88 | AS primer | TTCCTTCTCTTCCGCACCCGGCT | |
| SEQ ID NO: 89 | AS primer | TTCCTTCTCTTCCGCACCCATCT | |
| SEQ ID NO: 90 | AS primer | TTCCTTCTCTTCCGCACCCTGCT | |
| SEQ ID NO: 91 | Common | GTCTTCTCTGTTTCAGGGCATGAAC | |
| | primer | | |
| SEQ ID NO: 92 | probe | FTACTGGTGAAQAACACCGCAGCATGTP | |

F = FAM, Q = BHQ-2, P = phosphate

The allele-specific primers disclosed in this example achieved discrimination between the wild-type sequence and the L861Q mutation of ΔCₜ up to 57.5 cycles, depending on reaction conditions.

### Example 7

### Primers for Detecting Mutation S768I in the Human EGFR Gene

This mutation results from the nucleotide change 2301 G->T in the EGFR gene (SEQ ID NO: 1). Primers and probes for detecting the mutation are shown in Table 7. The mutation may be detected using an allele-specific primer selected from SEQ ID NOs: 93-101 and a common primer. Optionally, the common primer may be SEQ ID NO: 102. The amplification may be detected using a probe that hybridizes to the region between the allele-specific and a common primer. Optionally, the probe may be SEQ ID NO: 103. If the antisense strand is used, the mutation may be detected using an allele-specific primer selected from SEQ ID NOs: 104-106 and a common primer. Optionally, the common primer may be SEQ ID NO: 107. The amplification may be detected using a probe that hybridizes to the region between the allele-specific and a common primer. Optionally, the probe may be SEQ ID NO: 108.

TABLE 7
Primers and probes for detecting mutation S768I

| | | | |
|---|---|---|---|
| SEQ ID NO: 93 | AS primer | AGGAAGCCTACGTGATGGCCAT | |
| SEQ ID NO: 94 | AS primer | AGGAGGCCTACGTGATGGCCET | E = t-bb-dA |
| SEQ ID NO: 95 | AS primer | AGGAGGCCTACGTGATGGCEAT | E = t-bb-dC |
| SEQ ID NO: 96 | AS primer | AGGAGGCCTACGTGATGGECAT | E = t-bb-dC |
| SEQ ID NO: 97 | AS primer | AGGAGGCCTACGTGATGGCCGT | |
| SEQ ID NO: 98 | AS primer | AGGAGGCCTACGTGATGGCTAT | |
| SEQ ID NO: 99 | AS primer | AGGAGGCCTACGTGATGGCCTT | |
| SEQ ID NO: 100 | AS primer | AGGAGGCCTACGTGATGGCAAT | |
| SEQ ID NO: 101 | AS primer | AGGAGGCCTACGTGATGGACAT | |
| SEQ ID NO: 102 | Common | CCAATATTGTCTTTGTGTTCCCGGAC | |
| | primer | | |
| SEQ ID NO: 103 | probe | JCACGGTGGAGGTGAQGGCAGATGCP | |
| SEQ ID NO: 104 | AS primer | ACGTGGGGGTTGTCCACGA | |
| SEQ ID NO: 105 | AS primer | ATGTGGGGGTTGTCCACGA | |
| SEQ ID NO: 106 | AS primer | ATGTGGGGGTTGTCCGCGA | |
| SEQ ID NO: 107 | Common | ATCGCATTCATGCGTCTTCACC | |
| | primer | | |
| SEQ ID NO: 108 | probe | JAGTGTGGCTTCGCAQTGGTGGCCAGAAGGAP | |

J = Ja270, Q = BHQ-2, P = phosphate

The allele-specific primers disclosed in this example achieved discrimination between the wild-type sequence and the S768I mutation of ΔCₜ up to 71 cycles.

While the invention has been described in detail with reference to specific examples, it will be apparent to one skilled in the art that various modifications can be made within the scope of this invention. Thus the scope of the invention should not be limited by the examples described herein, but by the claims presented below.

What is claimed is:

1. An oligonucleotide comprising the primary sequence of oligonucleotides selected from SEQ ID NOs. 2, 10, 18, 27, 32, 51, 60, 71, 82, 93 and 104.

2. The oligonucleotide according to claim 1, further comprising at least one additional mismatch with the corresponding portion of SEQ ID NO: 1.

3. The oligonucleotide according to claim 1, further comprising at least one nucleotide with a base modified at the exocyclic amino group.

4. The oligonucleotide according to claim 3, wherein the nucleotide with a base modified at the exocyclic amino group is selected from tert-butyl-benzyl-deoxyadenine, tert-butyl-benzyl-deoxycytosine, methyl-deoxyadenine, methyl-deoxyxytosine, ethyl-deoxyadenine and ethyl-deoxycytosine.

5. An oligonucleotide selected from SEQ ID NOs. 3-7, 11-15, 19-24, 28, 29, 33-48, 52-57, 61-68, 72-79, 83-90, 94-101, 105 and 106.

6. An oligonucleotide selected from SEQ ID NOs. 8, 16, 25, 30, 49, 58, 69, 80, 91, 102 and 107.

7. An oligonucleotide selected from SEQ ID NOs. 9 and 109, 17 and 110, 26 and 111, 31 and 112, 50 and 113, 59 and 114, 70 and 115, 81 and 116, 92 and 117, 103 and 118 and 108 and 119.

8. The oligonucleotide according to claim 7, further comprising a detectable label.

9. A method of detecting mutations in the human epidermal growth factor receptor (EGFR) nucleic acid in a sample comprising:
(a) contacting the nucleic acid in the sample with the oligonucleotide of claim 1;
(b) incubating the sample under conditions allowing hybridization of the oligonucleotide to the target sequence within the EGFR nucleic acid;
(c) generating of the amplification product containing the target sequence within the EGFR nucleic acid; and
(d) detecting the presence of the amplified product thereby detecting the presence of the mutation in the EGFR nucleic acid.

10. The method of claim 9, wherein the nucleic acid in the sample is contacted with the oligonucleotide of claim 1.

11. The method of claim 9, wherein the amplification is performed by real-time PCR.

12. A method of determining whether a patient having a malignant tumor is likely to respond to EGFR inhibitors, comprising:
(a) contacting the nucleic acid in the sample from the patient with the oligonucleotide of claim 1;
(b) incubating the sample under conditions allowing hybridization of the oligonucleotide to the target sequence within the EGFR nucleic acid; and generation of the amplification product containing the target sequence within the EGFR nucleic acid;
(c) detecting the presence of the amplified product thereby detecting the presence of the mutation in the EGFR nucleic acid, wherein the presence of the mutation indicates that the patient is likely to respond to EGFR inhibitors.

13. The method of claim 12, wherein the amplification in step (b) and detection in step (c) are performed by real-time PCR.

14. The method of claim, 12 wherein said EGFR inhibitor is cetuximab, panitumumab, erlotinib or gefitinib.

15. A kit for detecting mutations in the human epidermal growth factor receptor (EGFR) gene comprising one or more pairs of oligonucleotides selected from pairs (a)-(k):
(a) an oligonucleotide of one of SEQ ID NOs: 2-7 and the oligonucleotide of SEQ ID NO: 8;
(b) an oligonucleotide of one of SEQ ID NOs: 10-15 and the oligonucleotide of SEQ ID NO: 16;
(c) an oligonucleotide of one of SEQ ID NOs: 18-24 and the oligonucleotide of SEQ ID NO: 25;
(d) an oligonucleotide of one of SEQ ID NOs: 27-29 and the oligonucleotide of SEQ ID NO: 30;
(e) an oligonucleotide of one of SEQ ID NOs: 32-48 and the oligonucleotide of SEQ ID NO: 49;
(f) an oligonucleotide of one of SEQ ID NOs: 51-57 and the oligonucleotide of SEQ ID NO: 58;
(g) an oligonucleotide of one of SEQ ID NOs: 60-68 and the oligonucleotide of SEQ ID NO: 69;
(h) an oligonucleotide of one of SEQ ID NOs: 71-79 and the oligonucleotide of SEQ ID NO: 80;
(i) an oligonucleotide of one of SEQ ID NOs: 82-90 and the oligonucleotide of SEQ ID NO: 91;
(j) an oligonucleotide of one of SEQ ID NOs: 93-101 and the oligonucleotide of SEQ ID NO: 102;
(k) an oligonucleotide of one of SEQ ID NOs: 104-106 and the oligonucleotide of SEQ ID NO: 107.

16. The kit of claim 15, further comprising an additional oligonucleotide as follows:
the kit with the pair of oligonucleotides (a) further comprising the oligonucleotide of SEQ ID NOS 9 and 109;
the kit with the pair of oligonucleotides (b) further comprising the oligonucleotide of SEQ ID NOS 17 and 110;
the kit with the pair of oligonucleotides (c) further comprising the oligonucleotide of SEQ ID NOS 26 and 111;
the kit with the pair of oligonucleotides (d) further comprising the oligonucleotide of SEQ ID NOS 31 and 112;
the kit with the pair of oligonucleotides (e) further comprising the oligonucleotide of SEQ ID NOS 50 and 113;
the kit with the pair of oligonucleotides (f) further comprising the oligonucleotide of SEQ ID NOS 59 and 114;
the kit with the pair of oligonucleotides (g) further comprising the oligonucleotide of SEQ ID NOS 70 and 115;
the kit with the pair of oligonucleotides (h) further comprising the oligonucleotide of SEQ ID NOS 81 and 116;
the kit with the pair of oligonucleotides (i) further comprising the oligonucleotide of SEQ ID NOS 92 and 117;
the kit with the pair of oligonucleotides (j) further comprising the oligonucleotide of SEQ ID NOS 103 and 118;
the kit with the pair of oligonucleotides (k) further comprising the oligonucleotide of SEQ ID NOS 108 and 119.

17. The kit of claim 15, wherein said additional oligonucleotide is labeled.

18. The kit of claim 15, further comprising nucleoside triphosphates, nucleic acid polymerase and buffers necessary for the function of the nucleic acid polymerase.

19. A reaction mixture for detecting mutations in the human epidermal growth factor receptor (EGFR) gene comprising one or more pairs of oligonucleotides selected from pairs (a)-(k):
an oligonucleotide of one of SEQ ID NOs: 2-7 and the oligonucleotide of SEQ ID NO: 8;
an oligonucleotide of one of SEQ ID NOs: 10-15 and the oligonucleotide of SEQ ID NO: 16;
an oligonucleotide of one of SEQ ID NOs: 18-24 and the oligonucleotide of SEQ ID NO: 25;
an oligonucleotide of one of SEQ ID NOs: 27-29 and the oligonucleotide of SEQ ID NO: 30;
an oligonucleotide of one of SEQ ID NOs: 32-48 and the oligonucleotide of SEQ ID NO: 49;
an oligonucleotide of one of SEQ ID NOs: 51-57 and the oligonucleotide of SEQ ID NO: 58;
an oligonucleotide of one of SEQ ID NOs: 60-68 and the oligonucleotide of SEQ ID NO: 69;
an oligonucleotide of one of SEQ ID NOs: 71-79 and the oligonucleotide of SEQ ID NO: 80;
an oligonucleotide of one of SEQ ID NOs: 82-90 and the oligonucleotide of SEQ ID NO: 91;
an oligonucleotide of one of SEQ ID NOs: 93-101 and the oligonucleotide of SEQ ID NO: 102;
an oligonucleotide of one of SEQ ID NOs: 104-106 and the oligonucleotide of SEQ ID NO: 107.

20. The reaction mixture of claim 22, further comprising an additional oligonucleotide as follows:
the reaction mixture with the pair of oligonucleotides (a) further comprising the oligonucleotide of SEQ ID NOS 9 and 109;
the reaction mixture with the pair of oligonucleotides (b) further comprising the oligonucleotide of SEQ ID NOS 17 and 110;
the reaction mixture with the pair of oligonucleotides (c) further comprising the oligonucleotide of SEQ ID NOS 26 and 111;
the reaction mixture with the pair of oligonucleotides (d) further comprising the oligonucleotide of SEQ ID NOS 31 and 112;
the reaction mixture with the pair of oligonucleotides (e) further comprising the oligonucleotide of SEQ ID NOS 50 and 113;
the reaction mixture with the pair of oligonucleotides (f) further comprising the oligonucleotide of SEQ ID NOS 59 and 114;
the reaction mixture with the pair of oligonucleotides (g) further comprising the oligonucleotide of SEQ ID NOS 70 and 115;
the reaction mixture with the pair of oligonucleotides (h) further comprising the oligonucleotide of SEQ ID NOS 81 and 116;
the reaction mixture with the pair of oligonucleotides (i) further comprising the oligonucleotide of SEQ ID NOS 92 and 117;
the reaction mixture with the pair of oligonucleotides (j) further comprising the oligonucleotide of SEQ ID NOS 103 and 118;
the reaction mixture with the pair of oligonucleotides (k) further comprising the oligonucleotide of SEQ ID NOS 108 and 119.
